(19) United States
(12) Patent Application Publication
(10) Pub. No.: US 2018/0133151 A1
(43) Pub. Date: May 17, 2018

(54) NEBULIZED TIOTROPIUM

(71) Applicant: GLENMARK SPECIALTY S.A., La Chaux-de-Fonds (CH)

(72) Inventors: Ulhas R Dhuppad, Nashik (IN); Franciscus Koppenhagen, Deerfield Beach, FL (US); Julianne Berry, Westfield, NJ (US); Sunil Chaudhari, Nashik (IN); Suresh Rajurkar, Nashik (IN); Chandrakant Dhatrak, Nashik (IN); Alkesh Kasliwal, Nanded (IN)

(21) Appl. No.: 15/815,336

(22) Filed: Nov. 16, 2017

(30) Foreign Application Priority Data
Nov. 16, 2016 (IN) 201621039057

(51) Int. Cl. A61K 9/00; A61K 31/439; A61K 47/02; A61M 11/00; A61M 15/00

(52) U.S. Cl. CPC A61K 9/0078; A61K 31/439; A61K 47/02; A61M 15/0065; A61M 11/005; A61M 15/001; A61M 11/003

(57) ABSTRACT

The present invention relates to therapeutic methods of administering tiotropium using a nebulizer. The present invention also relates to methods of treating inflammatory or obstructive airway diseases by administering a sterile nebulizable composition of tiotropium using a nebulizer.

This application claims the benefit of Indian Patent Application No. 201621039057, filed Nov. 16, 2016, which is hereby incorporated by reference.

### FIELD OF THE INVENTION

The present invention relates to therapeutic methods of administering tiotropium using a nebulizer. The present invention also relates to methods of treating inflammatory or obstructive airway diseases by administering a sterile nebulizable composition of tiotropium using a nebulizer.

### BACKGROUND OF THE INVENTION

Respiratory disorders include a number of airway diseases. Asthma and chronic obstructive airway disease (COPD) are among the most prevalent and life threatening conditions.

COPD is a chronic disorder that is characterized by loss of elasticity of the airways and air sacs, destruction of alveolar walls, inflammation of airways, and high mucus production in the airways. All of these effects lead to clogging of the airways making it difficult for the patient to breathe. Asthma, on the other hand, is a chronic disease involving airways of the lung that is characterized by coughing, wheezing, and shortness of breath.

Currently available treatment options include corticosteroids, beta agonists, anticholinergic agents, and expectorants.

Tiotropium is approved in the U.S. under the brand name Spiriva Respimat® as a propellant free metered dose inhaler. This product is approved with a “Respimat device”. This device is complex and requires the patient to co-ordinate breathing with the device.

There is a need for additional therapeutic options for the treatment of inflammatory or obstructive airway diseases, and in particular a need for more convenient and better therapeutic options for patients suffering from COPD and asthma.

### SUMMARY OF THE INVENTION

The present invention provides a method of treating an inflammatory or obstructive airway disease by administering a nebulizable composition of tiotropium or a pharmaceutically acceptable salt thereof using a nebulizer.

One embodiment relates to a method of delivering to a subject in need thereof a therapeutically effective amount of tiotropium or a pharmaceutically acceptable salt thereof (such as tiotropium bromide) using a nebulizer.

Another embodiment relates to a method of treating an inflammatory or obstructive airway disease comprising administering to a subject in need thereof, tiotropium or a pharmaceutically acceptable salt thereof (such as tiotropium bromide) using a nebulizer.

A further embodiment relates to a kit for treating an inflammatory or obstructive airway disease in a subject, comprising (i) an effective amount of tiotropium or a pharmaceutically acceptable salt thereof (such as tiotropium bromide), (ii) a nebulizer, and (iii) instructions for use in treating an inflammatory or obstructive airway disease.

Another embodiment relates to a method of delivering to a subject in need thereof a nebulizable composition of tiotropium or a pharmaceutically acceptable salt thereof (such as tiotropium bromide) using a nebulizer.

A further embodiment relates to a method of treating an inflammatory or obstructive airway disease comprising administering to a subject in need thereof, a nebulizable composition of tiotropium or a pharmaceutically acceptable salt thereof (such as tiotropium bromide) using a nebulizer.

Yet another embodiment relates to a kit for treating an inflammatory or obstructive airway disease in a subject, comprising (i) a nebulizable composition comprising an effective amount of tiotropium or a pharmaceutically acceptable thereof (such as tiotropium bromide), (ii) a nebulizer, and (iii) instructions for use in treating an inflammatory or obstructive airway disease.

In one embodiment, the nebulizable composition is a unit dose pharmaceutical solution for inhalation comprising tiotropium or a pharmaceutically acceptable salt thereof (such as tiotropium bromide). The nebulizable composition is preferably sterile.

In one preferred embodiment, the nebulizable composition is free, or substantially free, of preservatives including, but not limited to, quaternary ammonium preservatives, such as a benzalkonium salt (e.g., benzalkonium chloride).

Yet another embodiment is a sterile, unit dose nebulizable pharmaceutical solution for inhalation comprising tiotropium or a pharmaceutically salt thereof (such as tiotropium bromide), wherein the solution is free, or substantially free, of a complexing agent (such as ethylene diamine tetra-acetic acid (EDTA) and its salts). For example, the pharmaceutical solution may contain less than about 0.1% by weight of complexing agent (such as less than about 0.05%, less than about 0.02%, or less than about 0.008%), based on total weight of composition or solution. The pharmaceutical solution may also be free, or substantially free, of preservatives including, but not limited to, quaternary ammonium preservatives, such as a benzalkonium salt (e.g., benzalkonium chloride).

One embodiment is a nebulizable composition comprising

(i) tiotropium or a pharmaceutically acceptable salt thereof (such as tiotropium bromide); and
(ii) water
wherein the composition is free, or substantially free, of preservative and/or complexing agent. The nebulizable composition is preferably a solution.

Yet another embodiment is a sterile nebulizable pharmaceutical solution for inhalation via nebulization comprising tiotropium or a pharmaceutically salt thereof (such as tiotropium bromide), wherein the solution is free, or substantially free, of (a) EDTA or a salt thereof, (b) a benzalkonium salt, such as benzalkonium chloride, or (c) both.

Yet another embodiment is a pharmaceutical solution suitable for administration with a nebulizer consisting essentially of

(a) about 0.0005% to about 0.008% w/w tiotropium or a pharmaceutically acceptable salt thereof (such as tiotropium bromide),
(b) about 0% to about 0.008% w/w disodium EDTA;
(c) about 0% to about 0.9% w/w sodium chloride; and
(d) water,
based upon 100% total weight of the pharmaceutical solution, wherein the pH of the pharmaceutical solution is about 2 to about 4 (such as about 2.7).

In another embodiment, the nebulizable composition comprises about 0.002% to about 0.01% w/w tiotropium or any pharmaceutically acceptable salt thereof (such as tiotropium bromide), about 0% to about 0.01% w/w EDTA, and about 0.9% w/w sodium chloride, wherein the composition is free, or substantially free, of preservative and wherein the composition has a pH in the range of about 2.0 to about 4.0.

The nebulizable composition or solution provided herein is preferably sterile. The nebulizable composition or solution provided herein may have a long shelf life, i.e., it is stable during long term storage. The nebulizable composition may contain greater than about 80%, such as greater than about 85%, greater than about 90%, greater than about 95% or greater than about 98% of the initial amount of tiotropium or its salt in the composition after being stored for 3 or 6 months or 1, 2 or 3 years at 25° C. in a suitable LDPE container, cyclic olefin polymer container, cyclic olefin copolymer container, or glass container. The stability may be determined using Arrhenius kinetics.

The nebulizable compositions described herein may be delivered with a jet nebulizer, ultrasonic nebulizer, mesh nebulizer or a breath actuated nebulizer.

A further embodiment is a process for preparing an inhalation solution comprising tiotropium or a pharmaceutically acceptable salt thereof (e.g., for use in relieving bronchospasm associated with COPD). In one embodiment, the process comprises the steps of:
(a) dissolving tiotropium or its salt in water;
(b) optionally, adding one or more pharmaceutically acceptable excipients, such as a buffer, complexing agent, tonicity adjusting agent, or any combination thereof, to the solution of step (a);
(c) optionally, adjusting the pH of the solution (for example, the solution of step (a) or step (b)) with a pharmaceutically acceptable acid (e.g., to a pH of 2 to 4);
(d) optionally, filtering the solution (for example, with a 0.2 micron filter); and
(e) filling a suitable container with the solution.

In another embodiment, the process comprises the steps of:
(a) dissolving tiotropium or its salt in water;
(b) optionally, adding one or more pharmaceutically acceptable excipients such as a buffer, complexing agent, tonicity adjusting agent, or any combination thereof, to the solution of step (a);
(c) optionally, adjusting the pH of the solution (for example, the solution of step (a) or step (b)) with a pharmaceutically acceptable acid (e.g., to a pH of 2 to 4);
(d) filtering the solution (for example, with a 0.2 micron filter); and
(e) filling a suitable container with the solution.

Other objects, features and advantages of the present invention will be apparent to those of ordinary skill in the art in view of the following detailed description of the invention and accompanying drawings.

### DETAILED DESCRIPTION OF THE INVENTION

### Methods of Administration

One embodiment of the present invention is a method of administrating tiotropium or a pharmaceutically acceptable salt thereof to a subject in need thereof by administering to the subject the tiotropium or a pharmaceutically acceptable salt thereof with a nebulizer. The method can include administering a nebulizable composition (such as a nebulizable solution) described herein with the nebulizer.

Preferably, the method includes administering a therapeutically effective amount of tiotropium or a pharmaceutically acceptable salt thereof.

### Methods of Treatment

Another embodiment is a method of treating an inflammatory or obstructive airway disease (such as asthma and COPD) by administering to a subject in need thereof the tiotropium or a pharmaceutically acceptable salt thereof with a nebulizer. The method can include administering a nebulizable composition (such as a nebulizable solution) described herein with the nebulizer.

### Kit

Yet another embodiment is a kit for treating an inflammatory or obstructive airway disease in a subject comprising (i) a nebulizable composition comprising an effective amount of tiotropium or a pharmaceutically acceptable salt thereof, (ii) a nebulizer, and (iii) instructions for use in treating an inflammatory or obstructive airway disease.

### Tiotropium

A preferred salt of tiotropium is tiotropium bromide (for instance, in its monohydrate form). Tiotropium bromide has a molecular weight of 472.416 g/mol and the empirical formula C₁₉H₂₂BrNO₄S₂. Tiotropium bromide ((1+,2β,4β,7β)-7-[(hydroxydi-2-thienylacetyl)oxy]-9,9-dimethyl-3-oxa-9-azoniatricyclo[3.3.1.02,4]nonane bromide) is sparingly soluble in water and soluble in methanol. The established chemical structure of tiotropium bromide monohydrate is as follows:

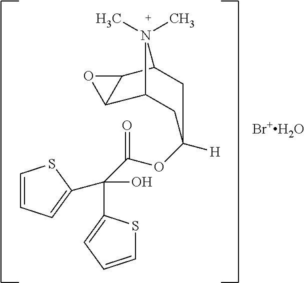

Salts of tiotropium include, but are not limited to, acid addition salts and base salts thereof, and any mixture thereof. Suitable salts of tiotropium include, but are not limited to, halide salts such as bromide, chloride and iodide salts. These and other salts are described, for example, in U.S. Pat. No. RE 39,820, which is hereby incorporated by reference in its entirety.

Different forms of Tiotropium include crystalline forms, amorphous form, monohydrate form and amorphous form. The preparation of tiotropium bromide monohydrate is described in U.S. Pat. No. 6,777,423, which is incorporated herein by reference in its entirety. Tiotropium and its salts can be administered to provide a bronchodilation effect and relief from symptoms associated with COPD. In one preferred embodiment, the nebulizable composition or solution includes tiotropium in the form of amorphous tiotropium bromide (such as amorphous anhydrous tiotropium bromide).

### Nebulizable Composition

Tiotropium may be provided in a variety of pharmaceutically acceptable vehicles, including, but not limited to, water or hydroalcoholic solution or any other aqueous solution comprising a pharmaceutically acceptable amount of an osmotic agent.

The nebulizable composition (such as a nebulizable solution) may include from about 0.001 mg to about 0.3 mg of tiotropium or its salt (such as tiotropium bromide), such as from about 0.010 mg to about 0.280 mg; about 0.020 mg to about 0.260 mg; about 0.025 mg to about 0.240 mg; about 0.005 mg to about 0.1 mg; about 0.005 mg to about 0.05 mg; about 0.01 mg to about 0.04 mg; about 0.02 to about 0.07 mg; about 0.04 mg to about 0.08 mg; about 0.04 mg to about 0.10 mg; about 0.05 mg to about 0.15 mg; about 0.10 mg to about 0.19 mg; about 0.15 mg to about 0.20 mg; about 0.20 mg to about 0.25 mg; or from about 0.26 mg to about 0.30 mg tiotropium or its salt (such as tiotropium bromide) per unit dosage of pharmaceutical composition or solution.

In another embodiment of the present invention, a therapeutically effective amount of tiotropium may include from about 0.0001% to about 0.030% by weight tiotropium bromide, including the following intermediate ranges of tiotropium bromide: about 0.0002 wt % to about 0.02 wt %; about 0.0003 wt % to about 0.01 wt %; about 0.0005 wt % to about 0.008 wt %; about 0.0002 wt % to about 0.001 wt %; about 0.001 wt % to about 0.005 wt %; about 0.006 wt % to about 0.010 wt %; about 0.011 wt % to about 0.015 wt %; about 0.016 wt % to about 0.020 wt %; about 0.021 wt % to about 0.025 wt %; or about 0.026 wt % to about 0.030 wt %.

In another embodiment, the amount of tiotropium or its salt (e.g., tiotropium bromide) in the nebulizable tiotropium composition is from about 1 μg to about 100 μg or from about 10 μg to about 80 μg, for example, about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg (based on the equivalent amount of tiotropium free base).

The nebulizable composition may comprise tiotropium bromide, and one or more pharmaceutically acceptable excipients. Suitable pharmaceutically acceptable excipients include, but are not limited to, pH adjusting agents, isotonicity agents, chelating agents, surfactants, anti-oxidants, and pharmaceutically acceptable vehicles.

The nebulizable composition may be substantially free, or free, of preservative (such as benzalkonium and salts thereof), and is preferably substantially benzalkonium chloride free. The term “substantially free” in connection with a preservative (for instance, “substantially benzalkonium chloride free”) denotes that the preservative is not present in an amount sufficient to materially act as a preservative for the nebulizable composition. A composition is “substantially benzalkonium chloride free” or “substantially free of benzalkonium chloride” when the amount of benzalkonium chloride is not an amount sufficient to materially act as a preservative for the nebulizable composition. In one embodiment, benzalkonium chloride may be present in a concentration less than 0.008% w/w based on total weight of the composition. In another embodiment, one or more preservatives may be present in a total concentration less than 0.008% w/w based on total weight of the composition.

Generally, nebulizable compositions contain a preservative such as benzalkonium chloride. A common problem with benzalkonium chloride is that it may cause paradoxic bronchoconstriction if the solution is administered repeatedly over short intervals, and frequent exposure to benzalkonium chloride may lead to occupational asthma. Another problem is that, when inhaled by patients, the benzalkonium chloride can cause dose-dependent bronchoconstriction. The nebulizable compositions of the present invention may be provided without benzalkonium chloride, thereby making them suitable for repeated administration over a short period of time. Also, administering a substantially benzalkonium chloride free nebulizable composition to a patient reduces the concomitant liability of adverse effects associated with benzalkonium chloride alone or in combination with other excipients and/or the active agents. It also negates the toxicity and other side effects associated with benzalkonium chloride.

The nebulizable composition may be free, or substantially free, of complexing agents such as ethylene diamine tetra-acetic acid (EDTA) and salts thereof. The absence of, or reduction in, the concentration of the additive EDTA and its salts helps to reduce the paradoxic effect associated with cough.

Suitable complexing agents include, but are not limited to, EDTA and salts thereof, such as edetate disodium. The nebulizable tiotropium composition may contain about 0.001% w/w complexing agent, about 0.01% w/w complexing agent, or about 0.02% complexing agent. The nebulizable tiotropium composition may contain about 0.01 mg/mL complexing agent, about 0.1 mg/mL complexing agent or about 0.2 mg/mL complexing agent.

The nebulizable tiotropium composition may contain about 0.001% w/w edetate disodium, about 0.01% w/w edetate disodium, or about 0.02% edetate disodium. The nebulizable tiotropium composition may contain about 0.01 mg/mL edetate disodium, about 0.1 mg/mL edetate disodium, or about 0.2 mg/mL edetate disodium.

A nebulizable composition may contain:

(i) tiotropium or its pharmaceutically acceptable salts thereof; and
(ii) water,
wherein the composition is free or substantially free of preservative, complexing agent, or both. In one preferred embodiment, the composition is free or substantially free of preservative and complexing agent.

The nebulizable composition may have a pH of from about 2.0 to about 6.0, such as from about 2.0 to about 4.0. A preferred pH range for tiotropium bromide compositions is from about 2.0 to about 4.5, such as from about 2.5 to 3.5 or from about 2.7 to about 3.2. Low pH levels, such as below about 3.2, are preferred for the long-term stability of the tiotropium salts in the nebulizable composition. In another embodiment, the nebulizable composition has a pH from about 2.2 to about 2.9. The pH may be adjusted by the addition of one or more pharmaceutically acceptable acids. Examples of suitable pharmaceutically acceptable acids include, but are not limited to, inorganic acids, such as hydrochloric acid, hydrobromic acid, nitric acid, sulfuric acid, and phosphoric acid, and any combination of any of the foregoing. Examples of other suitable pharmacologically acceptable acids include, but are not limited to, organic acids, such as ascorbic acid, citric acid, malic acid, maleic acid, tartaric acid, succinic acid, fumaric acid, acetic acid, formic acid, propionic acid, and any combination of any of the foregoing. In one embodiment, the pH is adjusted with 1N hydrochloric acid or 1N sulfuric acid. In another embodiment, the pH is adjusted with one or more organic acids selected from ascorbic acid, fumaric acid and citric acid. A preferred organic acid is citric acid. If desired, mixtures of the abovementioned acids may also be used, particularly in the case of acids which have other properties in addition to their acidifying properties, for instance those which act as flavorings or antioxidants, such as for example citric acid or ascorbic acid. The nebulizable composition may contain sodium citrate at a concentration of about 0.1 to about 1.0% (w/w) and citric acid at a concentration of about 0.1 to 1.0% (w/w) to control pH.

In certain embodiments, the pH of the tiotropium nebulizable composition may be from about 2.0 to about 6.0, preferably from about 2.0 to about 4.5, more preferably from about 2.5 to about 3.5, e.g., from about 2.5 to about 3.0. The tiotropium nebulizable composition may have a pH of about 2.5, about 2.6, about 2.7, about 2.8, about 2.9, about 3.0, about 3.1, about 3.2, about 3.3, about 3.4, or about 3.5. In one preferred embodiment, the pH is about 2.7.

The nebulizable composition may optionally include a buffer. General and biological buffers in the pH range of about 2.0 to about 8.0 include, but are not limited to, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, MES, BIS-TRIS, ADA, ACES, PIPES, MOPSO, BIS-TRIS PROPANE, BES, MOPS, TES, HEPES, DIPSO, MOBS, TAPSO, TRIZMA, HEPPSO, POPSO, TEA, EPPS, TRICINE, GLY-GLY, BICINE, HEPBS, TAPS, and AMPD buffers.

The osmolality of the nebulizable composition may be from about 200-500 mOsm/kg. The nebulizable composition may comprise a tonicity adjusting agent, such as an ionic salt (e.g., about 0.0001% w/v to about 264% w/v ionic salt). Suitable tonicity adjusting agents include, but are not limited to, ammonium carbonate, ammonium chloride, ammonium lactate, ammonium nitrate, ammonium phosphate, ammonium sulfate, ascorbic acid, bismuth sodium tartrate, boric acid, calcium chloride, calcium disodium edetate, calcium gluconate, calcium lactate, citric acid, dextrose, diethanolamine, dimethyl sulfoxide, edetate disodium, edetate trisodium monohydrate, fluorescein sodium, fructose, galactose, glycerin, lactic acid, lactose, magnesium chloride, magnesium sulfate, mannitol, polyethylene glycol, potassium acetate, potassium chlorate, potassium chloride, potassium iodide, potassium nitrate, potassium phosphate, potassium sulfate, propylene glycol, silver nitrate, sodium acetate, sodium bicarbonate, sodium biphosphate, sodium bisulfite, sodium borate, sodium bromide, sodium cacodylate, sodium carbonate, sodium chloride, sodium citrate, sodium iodide, sodium lactate, sodium metabisulfite, sodium nitrate, sodium nitrite, sodium phosphate, sodium propionate, sodium succinate, sodium sulfate, sodium sulfite, sodium tartrate, sodium thiosulfate, sorbitol, sucrose, tartaric acid, triethanolamine, urea, urethan, uridine, zinc sulfate, and any combination of any of the foregoing.

The nebulizable composition may include an osmotic adjusting agent. Suitable osmotic adjusting agents include, but are not limited to, sodium chloride, potassium chloride, zinc chloride, calcium chloride and any combination of any of the foregoing. Other osmotic adjusting agents include, but are not limited to, mannitol, glycerol, dextrose and any combination of any of the foregoing.

The pharmaceutically acceptable vehicle in the nebulizable composition includes water and optionally a cosolvent. Any cosolvent that is suitable for inhalation and capable of dissolving or solubilizing the tiotropium (or its salt) in the mixture of cosolvent and water can be used. Examples of suitable cosolvents include, for example, alcohols, ethers, hydrocarbons, and perfluorocarbons. Preferably, the cosolvent is a short chain polar alcohol. More preferably, the cosolvent is an aliphatic alcohol having from one to six carbon atoms, such as ethanol or isopropanol. A preferred cosolvent is ethanol. Non-limiting examples of suitable hydrocarbons include n-butane, isobutane, pentane, neopentane and isopentanes. Non-limiting examples of suitable ethers include dimethyl ether and diethyl ether. Non-limiting examples of suitable perfluorocarbons include perfluoropropane, perfluorobutane, perfluorocyclobutane, and perfluoropentane.

Suitable surfactants include, but are not limited to, C₅₋₂₀-fatty alcohols, C₅₋₂₀-fatty acids, C₅₋₂₀-fatty acid esters, lecithin, glycerides, propylene glycol esters, polyoxyethylenes, polysorbates, sorbitan esters, carbohydrates, and any combination of any of the foregoing. C₅₋₂₀-fatty acids, propylene glycol diesters of the C₅₋₂₀-fatty acids, triglycerides of the C₅₋₂₀-fatty acids, and sorbitans of the C₅₋₂₀-fatty acids are preferred. In one preferred embodiment, the surfactant is selected from oleic acid, sorbitan mono-, di- or trioleates, and any combination of any of the foregoing.

Suitable antioxidants include, but are not limited to, ascorbic acid, vitamin A, vitamin E, tocopherols, and any combination of any of the foregoing.

The volume of the composition may be from about 0.1 ml to about 5 ml, such as from about 1 ml to about 3 ml, or from about 1.5 ml to about 2.5 ml. In another embodiment, the volume of the composition is from about 0.05 ml to about 1.0 ml; such as from about 0.1 ml to about 0.9 ml; from about 0.1 ml to about 0.8 ml; from about 0.1 ml to about 0.7 ml; from about 0.1 ml to about 0.6 ml; from about 0.1 ml to about 0.5 ml; from about 0.1 ml to about 0.4 ml; from about 0.1 ml to about 0.3 ml; or from about 0.1 ml to about 0.2 ml.

A nebulizable composition suitable for administration with a nebulizer consists essentially of

(a) about 0.0005% to about 0.008% w/w tiotropium or a pharmaceutically acceptable salt thereof (such as tiotropium bromide),
(b) about 0% to about 0.008% w/w disodium EDTA;
(c) about 0% to about 0.9% w/w sodium chloride; and
(d) water,
based upon 100% total weight of the nebulizable solution, wherein the pH of the composition is about 2 to about 4 (such as about 2.7).

The nebulizable composition may comprise about 0.002% to about 0.01% w/w tiotropium or a pharmaceutically acceptable salt thereof (such as tiotropium bromide), about 0% to about 0.01% w/w EDTA, and about 0.9% w/w sodium chloride, wherein the composition is substantially free of preservative such as benzalkonium chloride and wherein the composition has a pH in the range of about 2.0 to about 4.0.

In one embodiment, the nebulizable composition comprises (a) about 10 to about 80 μg tiotropium bromide, (b) about 0.9% w/w sodium chloride, (c) about 0.001% w/w disodium edetate, (d) hydrochloric acid, and (e) water. The composition preferably has a pH of about 2.7. In one embodiment, the volume of the nebulizable composition is 2 mL. In another embodiment, the nebulizable composition comprises (a) about 10 to about 80 μg tiotropium bromide, (b) about 18,000 μg of sodium chloride, (c) 20 μg of disodium edetate, (d) hydrochloric acid, and (e) water. The volume of the composition may also be from about 0.1 ml to about 5 ml, such as from about 1 ml to about 3 ml, or from about 1.5 ml to about 2.5 ml. In another embodiment, the volume of the composition is from about 0.05 ml to about 1.0 ml; such as from about 0.1 ml to about 0.9 ml; from about 0.1 ml to about 0.8 ml; from about 0.1 ml to about 0.7 ml; from about 0.1 ml to about 0.6 ml; from about 0.1 ml to about 0.5 ml; from about 0.1 ml to about 0.4 ml; from about 0.1 ml to about 0.3 ml; or from about 0.1 ml to about 0.2 ml.

In one embodiment, the nebulizable composition comprises (a) about 10 to about 80 μg tiotropium bromide, (b) about 0.9% w/w sodium chloride, (c) about 0.01% w/w disodium edetate, (d) hydrochloric acid, and (e) water. The composition preferably has a pH of about 2.7. In one embodiment, the volume of the nebulizable composition is 2 mL. In another embodiment, the nebulizable composition comprises (a) about 10 to about 80 μg tiotropium bromide, (b) about 18,000 μg of sodium chloride, (c) 200 μg of disodium edetate, (d) hydrochloric acid, and (e) water. The volume of the composition may also be from about 0.1 ml to about 5 ml, such as from about 1 ml to about 3 ml, or from about 1.5 ml to about 2.5 ml. In another embodiment, the volume of the composition is from about 0.05 ml to about 1.0 ml; such as from about 0.1 ml to about 0.9 ml; from about 0.1 ml to about 0.8 ml; from about 0.1 ml to about 0.7 ml; from about 0.1 ml to about 0.6 ml; from about 0.1 ml to about 0.5 ml; from about 0.1 ml to about 0.4 ml; from about 0.1 ml to about 0.3 ml; or from about 0.1 ml to about 0.2 ml.

In another embodiment, the nebulizable composition comprises (a) about 10 to about 80 μg tiotropium bromide, (b) about 0.9% w/w sodium chloride, (c) about 0.02% w/w disodium edetate, (d) hydrochloric acid, and (e) water. The composition preferably has a pH of about 2.7. In one embodiment, the volume of the nebulizable composition is 2 mL. In another embodiment, the nebulizable composition comprises (a) about 10 to about 80 μg tiotropium bromide, (b) about 18,000 μg of sodium chloride, (c) 400 μg of disodium edetate, (d) hydrochloric acid, and (e) water. The volume of the composition may also be from about 0.1 ml to about 5 ml, such as from about 1 ml to about 3 ml, or from about 1.5 ml to about 2.5 ml. In another embodiment, the volume of the composition is from about 0.05 ml to about 1.0 ml; such as from about 0.1 ml to about 0.9 ml; from about 0.1 ml to about 0.8 ml; from about 0.1 ml to about 0.7 ml; from about 0.1 ml to about 0.6 ml; from about 0.1 ml to about 0.5 ml; from about 0.1 ml to about 0.4 ml; from about 0.1 ml to about 0.3 ml; or from about 0.1 ml to about 0.2 ml.

In another embodiment, the nebulizable composition comprises (a) about 10 to about 80 μg tiotropium bromide, (b) about 0.9% w/w sodium chloride, (c) about 0.05% w/w disodium edetate, (d) hydrochloric acid, and (e) water. The composition preferably has a pH of about 2.7. In one embodiment, the volume of the nebulizable composition is 2 mL. The volume of the composition may also be from about 0.1 ml to about 5 ml, such as from about 1 ml to about 3 ml, or from about 1.5 ml to about 2.5 nil. In another embodiment, the volume of the composition is from about 0.05 ml to about 1.0 ml; such as from about 0.1 ml to about 0.9 ml; from about 0.1 ml to about 0.8 ml; from about 0.1 ml to about 0.7 ml; from about 0.1 ml to about 0.6 ml; from about 0.1 ml to about 0.5 ml; from about 0.1 ml to about 0.4 ml; from about 0.1 ml to about 0.3 ml; or from about 0.1 ml to about 0.2 ml.

Yet another embodiment is a method of treating an inflammatory or obstructive airway disease, such as COPD, comprising administering via a vibrating mesh nebulizer a nebulizable composition, where the nebulizable composition comprises (a) about 10 to about 80 μg tiotropium bromide, (b) about 0.9% w/w sodium chloride, (c) about 0.001% w/w disodium edetate, (d) hydrochloric acid, and (e) water. The composition preferably has a pH of about 2.7. In one embodiment, the volume of the nebulizable composition is 2 mL. In another embodiment, the nebulizable composition comprises (a) about 10 to about 80 μg tiotropium bromide, (b) about 18,000 μg of sodium chloride, (c) 20 μg of disodium edetate, (d) hydrochloric acid, and (e) water. The volume of the composition may also be from about 0.1 ml to about 5 ml, such as from about 1 ml to about 3 ml, or from about 1.5 ml to about 2.5 ml. In another embodiment, the volume of the composition is from about 0.05 ml to about 1.0 ml; such as from about 0.1 ml to about 0.9 ml; from about 0.1 ml to about 0.8 ml; from about 0.1 ml to about 0.7 ml; from about 0.1 ml to about 0.6 ml; from about 0.1 ml to about 0.5 ml; from about 0.1 ml to about 0.4 ml; from about 0.1 ml to about 0.3 ml; or from about 0.1 ml to about 0.2 ml.

Yet another embodiment is a method of treating an inflammatory or obstructive airway disease, such as COPD, comprising administering via a vibrating mesh nebulizer a nebulizable composition, where the nebulizable composition comprises (a) about 10 to about 80 μg tiotropium bromide, (b) about 0.9% w/w sodium chloride, (c) about 0.01% w/w disodium edetate, (d) hydrochloric acid, and (e) water. The composition preferably has a pH of about 2.7. In one embodiment, the volume of the nebulizable composition is 2 mL. In another embodiment, the nebulizable composition comprises (a) about 10 to about 80 μg tiotropium bromide, (b) about 18,000 μg of sodium chloride, (c) 200 μg of disodium edetate, (d) hydrochloric acid, and (e) water. The volume of the composition may also be from about 0.1 ml to about 5 ml, such as from about 1 ml to about 3 ml, or from about 1.5 ml to about 2.5 ml. In another embodiment, the volume of the composition is from about 0.05 ml to about 1.0 ml; such as from about 0.1 ml to about 0.9 ml; from about 0.1 ml to about 0.8 ml; from about 0.1 ml to about 0.7 ml; from about 0.1 ml to about 0.6 ml; from about 0.1 ml to about 0.5 ml; from about 0.1 ml to about 0.4 ml; from about 0.1 ml to about 0.3 ml; or from about 0.1 ml to about 0.2 ml.

Yet another embodiment is a method of treating an inflammatory or obstructive airway disease, such as COPD, comprising administering via a vibrating mesh nebulizer a nebulizable composition, where the nebulizable composition comprises (a) about 10 to about 80 μg tiotropium bromide, (b) about 0.9% w/w sodium chloride, (c) about 0.02% w/w disodium edetate, (d) hydrochloric acid, and (e) water. The composition preferably has a pH of about 2.7. In one embodiment, the volume of the nebulizable composition is 2 mL. In another embodiment, the nebulizable composition comprises (a) about 10 to about 80 μg tiotropium bromide, (b) about 18,000 μg of sodium chloride, (c) 400 μg of disodium edetate, (d) hydrochloric acid, and (e) water. The volume of the composition may also be from about 0.1 ml to about 5 ml, such as from about 1 ml to about 3 ml, or from about 1.5 ml to about 2.5 ml. In another embodiment, the volume of the composition is from about 0.05 ml to about 1.0 ml; such as from about 0.1 ml to about 0.9 ml; from about 0.1 ml to about 0.8 ml; from about 0.1 ml to about 0.7 ml; from about 0.1 ml to about 0.6 ml; from about 0.1 ml to about 0.5 ml; from about 0.1 ml to about 0.4 ml; from about 0.1 ml to about 0.3 ml; or from about 0.1 ml to about 0.2 ml.

The nebulizable composition provided herein may have a long shelf life, i.e., it is stable during long term storage. The nebulizable composition may contain greater than about 80%, such as greater than about 85%, greater than about 90%, greater than about 95% or greater than about 98% of the initial amount of tiotropium or its salt in the composition after being stored for 3 or 6 months or 1, 2 or 3 years at 25° C. in a suitable LDPE container, cyclic olefin polymer container, cyclic olefin copolymer container, or glass container. The stability may be determined using Arrhenius kinetics.

The nebulizable compositions can be contained in a prefilled container.

The nebulizable composition may be contained in a unit-dose, low-density polyethylene (LDPE) container, polypropylene container, or a cyclic polyolefin container. Each unit-dose container may be disposed in a foil pouch, and each foil pouch may contain 2 or more unit-dose containers. Each foil pouch containing the unit dose container may be disposed in a shelf carton.

### Nebulizer Device

Suitable nebulizer devices for use with the nebulizable composition include jet nebulizers, ultrasonic nebulizers, mesh nebulizers and breath actuated nebulizers, more preferably, mesh nebulizers.

Jet nebulizers work by passing an air stream through a venturi nozzle and nebulizing the liquid. The negative pressure created by the gas results in increased velocity of the liquid coming in from the reservoir. There are baffle plates which are arranged behind the jet openings to further reduce the droplet size. The advantages of the jet nebulizers are that they are easy to handle and have a robust design. However, jet nebulizers are noisy and have longer nebulization times. Commercially available jet nebulizer include Acorn-I®, Acorn-II®, AquaTower®, AVA-NEB®, Cirrhus®, Dart®, DeVilbiss 646®, Downdraft®, Fan Jet®, MB-5®, Misty Neb®, PART LC JET®, PART-JET®, Salter 8900®, Sidestream®, Updraft-II®, and Whisper Jet®.

Ultrasonic nebulizers work on a piezoelectric crystal technology. Briefly, these nebulizers contain a piezoelectric crystal which vibrates at high frequencies to produce an aerosol. Ultrasonic nebulizers however suffer from limitations such as heating up of the devices (which renders them unsuitable for thermo labile active agents), large residual volumes, and the inability to nebulize viscous solutions. Commercially available ultrasonic nebulizers include DeVilbiss-Pulmosonic®, Rho\hat{n}e Poulenc-Rorer-Fisoneb®, Omron NE-U1®, and Beurer Nebulizer IH30®.

Mesh nebulizers are relatively new devices and pose many advantages over the jet and the ultrasonic nebulizers. This nebulizer typically uses a mesh plate or an aperture plate with precisely drilled holes whose diameter controls the size of the aerosolized droplets. A piezoelectric element is in contact with the mesh plate which vibrates upon supply of electric current. The nebulizable composition is in direct contact with the mesh which upon vibration converts the liquid into aerosol droplets which can be inhaled by the patient. A dense aerosol is produced as a result of the vibrations which can be administered to a patient. Mesh nebulizers can be either static mesh or vibrating mesh nebulizers. Static mesh nebulizers push the liquid through a static mesh under the vibrations generated by an ultrasonic transducer. Vibrating mesh nebulizers use mesh vibrations or mesh deformations to generate an aerosol. Commercially available mesh nebulizers include Micro Air® from Omron, i-Neb® from Respironics, Akita® from Activaero, AeroNeb Go®, AeroNeb Pro®, AeroNeb Solo® from Aerogen and Aerovect Rx® from Aerovect.

A respirable dose delivery rate of about 0.25 μg/min to about 20 μg/min may be achieved by the nebulizable composition when administered by a nebulizer device.

The nebulizable composition administered by a vibrating mesh nebulizer may provide an aerosol having a Geometric Standard Deviation of emitted droplet size distribution of the nebulizable composition of about 1 to about 3.

The nebulizable composition administered by a vibrating mesh nebulizer may provide an aerosol having a Mass Median Aerodynamic Diameter of droplet size of the nebulizable composition of about 2.5 micron to about 10.5 micron.

A method of administering a nebulizable composition of tiotropium by a vibrating mesh nebulizer, wherein said composition comprises about 1 mcg to about 100 mcg tiotropium, wherein said nebulization composition when administered by the said vibrating mesh nebulizer provides an aerosol having a mass median aerodynamic diameter of about 2.5 microns to about 10.5 microns and a geometric standard deviation of emitted droplet distribution of about 1 to about 3.

The nebulizable composition administered by a vibrating mesh nebulizer may provide a fine particle dose which is not less than 10%.

The fine particle fraction obtained by administering the nebulizable composition in a vibrating mesh nebulizer may be about 10% to about 60%.

The nebulizable compositions of the present invention when administered by a vibrating mesh nebulizer to a Breath simulator (BRS) exhibit a delivered dose of about 10% to about 70%.

The nebulizable compositions of the present invention when administered by a vibrating mesh nebulizer exhibit a droplet size distribution wherein the D10 is not more than about 5 micron, the D50 is not more than about 10 micron, the D90 is not more than about 20 micron, and the Span [(D90−D10)/D50] is not more than about 5.

The time taken to nebulize the nebulizable composition from the nebulizer device may be about 1 to about 15 minutes.

### Definitions

Throughout this specification it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

The term “subject” includes, but is not limited to, humans.

To treat indications with a therapeutic agent, an “effective amount” of a therapeutic agent will be recognized by clinicians and persons of ordinary skill in the art, and includes an amount effective to treat, reduce, alleviate, ameliorate, eliminate or prevent one or more symptoms of the condition sought to be treated, or alternately, the condition sought to be avoided, or to otherwise produce a clinically recognizable favorable change in the condition or its effects.

“Respirable dose delivery rate” is the speed at which a respirable dose of the drug is nebulized, administered, and delivered to a patient's lungs.

The term “Geometric Standard Deviation” is the geometric breadth of the best-fitted log-normal function to the particle size data.

The term “Mass Median Aerodynamic Diameter” is the median aerodynamic size of a plurality of particles, typically in a polydisperse population. The “aerodynamic diameter” is the diameter of a unit density sphere having the same settling velocity, generally in air, as a powder and is therefore a useful way to characterize an aerosolized powder or other dispersed particle or particle formulation in terms of its settling behavior. The aerodynamic diameter encompasses particle or particle shape, density, and physical size of the particle or particle. MMAD is determined herein by cascade impaction, unless the context indicates otherwise.

The term “Fine particle dose” is the dose, expressed in μg or the percentage of the total dose, of the aerosolized drug particles with an aerodynamic diameter <5 micron.

The term “Fine particle fraction” is the ratio of Fine particle dose to the total recovered dose.

The term “D10” is the particle diameter value that 10% of the population of particles lies below.

The term “D50” is the particle diameter value that 50% of the population lies below and 50% of the population lies above.

The term “D90” is the particle diameter value that 90% of the population lies below.

The term “by weight” indicates “% w/w” of the total weight of the nebulizable composition.

### EXAMPLES

The following examples further illustrate the invention, but are not limiting.

### Example 1

| | | Quantity (μg/2 ml) | | | |
|---|---|---|---|---|---|
| S.N | Ingredients | 10 μg/2 ml | 20 μg/2 ml | 40 μg/2 ml | 80 μg/2 ml |
| 1 | Tiotropium | 10 | 20 | 40 | 80 |
| | bromide IH | | | | |
| 2 | Sodium | 18000 | 18000 | 18000 | 18000 |
| | chloride USP | | | | |
| 3 | Disodium | 20 | 20 | 20 | 20 |
| | edetate USP | | | | |
| 4 | Hydrochloric | q.s. to pH | q.s. to pH | q.s. to pH | q.s. to pH |
| | acid USP as | 2.7 | 2.7 | 2.7 | 2.7 |
| | 0.5N HCl | | | | |
| | solution | | | | |
| 5 | Water for | q.s. to 2 mL | q.s. to 2 mL | q.s. to 2 mL | q.s. to 2 mL |
| | injection USP | | | | |

### Process:

1. Collect 85% of batch quantity water for injection (WFI) in a manufacturing vessel. Cool WFI to 20-25° C.
2. Add and dissolve to it a batch quantity of sodium chloride under stirring. Check clarity of the solution.
3. Add and dissolve to it a batch quantity of disodium edetate under stirring. Check clarity of the solution.
4. Check pH and adjust pH to 2.7 using 0.5N HCl solution.
5. Add and dissolve to it a batch quantity of tiotropium bromide anhydrous under stirring. Check clarity of the solution.
6. Make up volume of bulk. Send bulk sample for analysis.
7. Filter bulk through 0.22 μm PVDF filter.
8. Fill bulk in LDPE vials.

The contents of the compositions illustrated above were poured into the reservoir of a vibrating mesh nebulizer, such as Omron Micro Air NE-U22. The compositions were then evaluated using a Next Generation Impactor (NGI) device. NGI device mimics several components of the respiratory tract. The data generated from an NGI device is a representation of the proportion of the drug getting deposited into various portions of the respiratory tract. It provides an estimate of the proportion of the swallowed vs inhaled drug, drug remaining in device and drug reaching the target sites. Evaluation measures include, but are not limited to, Fine particle fraction (FPF), Fine particle dose (FPD), Mass Median Aerodynamic Diameter (MMAD), Geometric Standard Deviation (GSD) etc.

The following table is the performance of the nebulizable compositions in a vibrating mesh nebulizer, such as Omron Micro Air NE-U22.

| | # | | | | | | | |
|---|---|---|---|---|---|---|---|---|
| | 10 μg/2 mL | | 20 μg/2 mL | | 40 μg/2 mL | | 80 μg/2 mL | |
| | % | μg | % | μg | % | μg | % | μg |
| Delivered dose by NGI | 74 | 7.4 | 78 | 15.66 | 82.04 | 32.82 | 89.91 | 71.93 |
| Mass Balance | 95.31 | 9.53 | 91.99 | 18.4 | 100.59 | 40.23 | 97.43 | 77.95 |
| FPD | 24.27 | 2.43 | 26.14 | 5.23 | 26.55 | 10.62 | 28.32 | 22.66 |
| FPF | 32.8 | | 33.52 | | 32.37 | | 31.5 | |
| ISM (Stage 1 To MOC) | | 7.31 | | 15.41 | | 31.24 | | 69.36 |

| MMAD (μm) | 6.637 | 6.579 | 6.547 | 6.749 |
|---|---|---|---|---|
| GSD | 1.958 | 1.902 | 1.879 | 1.860 |
| Nebulization time (mins) | 8 | 4.5 | 4.5-5 | 4 |
| Residue in cup (%) | ~27 | ~21 | ~26 | ~22 |

### Example 2

| | | Quantity (μg/2 ml) | | | |
|---|---|---|---|---|---|
| S.N | Ingredients | 10 μg/2 ml | 20 μg/2 ml | 40 μg/2 ml | 80 μg/2 ml |
| 1 | Tiotropium | 12.04 | 24.07 | 48.14 | 96.29 |
| | bromide IH | | | | |
| 2 | Sodium | 18000.00 | 18000.00 | 18000.00 | 18000.00 |
| | chloride USP | | | | |
| 3 | Disodium | 400.00 | 400.00 | 400.00 | 400.00 |
| | edetate USP | | | | |
| 4 | Hydrochloric | q.s. to adjust | q.s. to adjust | q.s. to adjust | q.s. to adjust |
| | acid USP as | pH | pH | pH | pH |
| | 0.5N HCl | | | | |
| | solution | | | | |
| 5 | Water for | q.s. to 2 mL | q.s. to 2 mL | q.s. to 2 mL | q.s. to 2 mL |
| | injection USP | | | | |

### Process:

1. Collect 85% of batch quantity water for injection. Cool water for injection to 20-25° C.
2. Add & dissolve to it sodium chloride under stirring. Check clarity of the solution.
3. Add & dissolve to it disodium edetate under stirring. Check clarity of the solution.
4. Check pH & adjust pH to 2.7 using 1N HCl solution.
5. Add & dissolve to it tiotropium bromide anhydrous under stirring. Check clarity of the solution.
6. Make up volume of bulk.
7. Filter bulk through 0.22μ PVDF filter.
8. Filling of bulk in LDPE vials.
   The contents of the compositions illustrated in Example 2 above were poured into the reservoir of different vibrating mesh nebulizers and the delivery was then evaluated using a Next Generation Impactor (NGI) device.

| | # | | | | | |
|---|---|---|---|---|---|---|
| | 10 μg/2 mL | | | | | |
| | Device 1 | | Device 2 | | Device 3 | |
| | μg | % | μg | % | μg | % |
| Delivered dose | 7.73 | 98.1 | 8.75 | 99.10 | 9.38 | 93.80 |
| by NGI | | | | | | |
| Mass Balance | 9.81 | 77.30 | 9.91 | 87.50 | 9.82 | 98.20 |
| FPD | 4.13 | 41.29 | 3.46 | 34.57 | 4.36 | 43.60 |
| FPF | — | 53.14 | — | 39.51 | — | 46.48 |

| | | | |
|---|---|---|---|
| MMAD (μm) | 4.62 | 5.77 | 5.13 |
| GSD | 1.99 | 1.93 | 1.68 |
| Nebulization | 3 minutes | 7 minutes | 10 minutes |
| time (mins) | | | |

| | # | | | | | | | |
|---|---|---|---|---|---|---|---|---|
| | 40 μg/2 mL | | | | | | | |
| | Device 1 | | Device 2 | | Device 3 | | Device 4 | |
| | μg | % | μg | % | μg | % | μg | % |
| Delivered dose | 28.74 | 92.60 | 34.06 | 96.48 | 36.21 | 90.53 | 18.95 | 47.38 |
| by NGI | | | | | | | | |
| Mass Balance | 37.04 | 71.85 | 38.59 | 85.15 | 37.62 | 94.05 | 38.96 | 97.40 |
| FPD | 16.06 | 40.15 | 13.83 | 34.57 | 18.26 | 45.65 | 11.31 | 28.26 |
| FPF | — | 55.89 | — | 40.60 | — | 50.43 | — | 59.66 |

| | | | | |
|---|---|---|---|---|
| MMAD (μm) | 4.45 | 5.67 | 4.87 | 3.84 |
| GSD | 2.01 | 1.88 | 1.72 | 2.39 |
| Nebulization | 3 minutes | 7 minutes | 10 minutes | 4 minutes |
| time (mins) | | | | |

The contents of the compositions illustrated in Example 2 above were poured into the reservoir of different vibrating mesh nebulizers and the delivery was then evaluated using a Breath simulator (BRS) device.

| | |
|---|---|
| Total delivered | |
| dose | Cup retention |

| Strength | Device | μg | % | μg | % |
|---|---|---|---|---|---|
| 10 μg/ | Device 1 | 3.15 | 31.50 | 3.20 | 31.20 |
| 2 ml | Device 2 | 2.9 | 29.4 | 2.3 | 23.4 |
| | Device 3 | 3.65 | 36.50 | 2.28 | 22.8 |
| 40 μg/ | Device 1 | 12.70 | 31.70 | 11.40 | 28.60 |
| 2 ml | Device 2 | 11.2 | 28.0 | 8.3 | 20.7 |
| | Device 3 | 14.4 | 36 | 6.38 | 16.0 |
| | Device 4 | 5.5 | 13.7 | 26.2 | 65.4 |

The nebulizable compositions were analyzed for the droplet size distribution data. The results from three different vibrating mesh nebulizers is given below:

### Device 1:

| # | D10 (μm) | D50 (μm) | D90 (μm) | SPAN |
|---|---|---|---|---|
| 1 | 1.818 | 4.197 | 8.69 | 1.638 |
| 2 | 1.821 | 4.175 | 8.63 | 1.630 |
| 3 | 1.843 | 4.304 | 8.97 | 1.655 |
| 4 | 1.815 | 4.227 | 8.83 | 1.660 |
| 5 | 1.824 | 4.240 | 8.82 | 1.651 |
| 6 | 1.815 | 4.223 | 8.81 | 1.656 |
| 7 | 1.822 | 4.194 | 8.69 | 1.637 |
| 8 | 1.829 | 4.219 | 8.76 | 1.642 |
| 9 | 1.834 | 4.326 | 9.12 | 1.684 |
| 10 | 1.823 | 4.271 | 8.94 | 1.667 |
| MEAN | 1.824 | 4.238 | 8.83 | 1.652 |
| % RSD | 0.48 | 1.15 | 1.69 | 0.98 |
| Maximum | 1.843 | 4.326 | 9.12 | 1.684 |
| Minimum | 1.815 | 4.175 | 8.63 | 1.630 |

### Device 2:

| # | D10 (μm) | D50 (μm) | D90 (μm) | SPAN |
|---|---|---|---|---|
| 1 | 2.564 | 5.915 | 11.75 | 1.553 |
| 2 | 2.518 | 5.929 | 11.90 | 1.582 |
| 3 | 2.454 | 5.831 | 11.79 | 1.601 |
| 4 | 2.253 | 5.554 | 11.33 | 1.635 |
| 5 | 2.335 | 5.646 | 11.43 | 1.611 |
| 6 | 2.252 | 5.482 | 11.15 | 1.624 |
| 7 | 2.313 | 5.608 | 11.39 | 1.619 |
| 8 | 2.342 | 5.644 | 11.45 | 1.614 |
| 9 | 2.265 | 5.497 | 11.22 | 1.629 |
| 10 | 2.100 | 5.306 | 11.13 | 1.701 |
| MEAN | 2.340 | 5.641 | 11.45 | 1.617 |
| % RSD | 5.95 | 3.55 | 2.39 | 2.37 |
| Maximum | 2.564 | 5.929 | 11.90 | 1.701 |
| Minimum | 2.100 | 5.306 | 11.13 | 1.553 |

### Device 3:

| # | D10 (μm) | D50 (μm) | D90 (μm) | SPAN |
|---|---|---|---|---|
| 1 | 2.834 | 6.991 | 14.71 | 1.698 |
| 2 | 2.550 | 5.717 | 11.66 | 1.593 |
| 3 | 2.616 | 5.795 | 11.64 | 1.558 |
| 4 | 2.346 | 5.846 | 12.53 | 1.742 |
| 5 | 2.452 | 6.525 | 14.23 | 1.805 |
| 6 | 2.470 | 5.508 | 11.06 | 1.559 |
| 7 | 2.572 | 5.834 | 11.90 | 1.599 |
| 8 | 2.634 | 6.121 | 12.71 | 1.645 |
| 9 | 2.496 | 6.296 | 13.57 | 1.759 |
| 10 | 2.326 | 5.460 | 11.27 | 1.638 |
| MEAN | 2.530 | 6.009 | 12.53 | 1.660 |
| % RSD | 5.88 | 7.97 | 10.11 | 5.26 |
| Maximum | 2.834 | 6.991 | 14.71 | 1.805 |
| Minimum | 2.326 | 5.460 | 11.06 | 1.558 |

### Abbreviations

NGI=next generation impactor
MOC=micro-orifice collector
FPD=fine particle dose
FPF=fine particle fraction
ISM=impactor sized mass
MMAD=mass median aerodynamic diameter
GSD=geometric standard deviation

1. A method for administering tiotropium bromide comprising administering via a vibrating mesh nebulizer a nebulizable composition to generate an aerosol at a respirable dose delivery rate of about 0.25 μg/min to about 20 μg/min.

2. A method for administering tiotropium bromide comprising administering via a vibrating mesh nebulizer a nebulizable composition to generate an aerosol having one or more of
(i) a geometric standard deviation of emitted droplet size distribution of the nebulizable composition of about 1 to about 3,
(ii) a mass median aerodynamic diameter of droplet size of the nebulizable composition of about 2.5 micron to about 10.5 micron, or
(iii) any combination of any of the foregoing.

3. The method of claim 2, wherein the aerosol has a droplet size distribution with a D10 of not more than about 5 microns, a D50 of not more than about 10 microns, a D90 of not more than about 20 microns, a Span [(D90−D10)/D50] of not more than about 5, or any combination of any of the foregoing,

4. The method of claim 2, wherein the aerosol has a fine particle dose of not less than 10%.

5. The method of claim 2, wherein the aerosol has a fine particle fraction of about 10% to about 60%.

6. The method of claim 2, wherein the nebulizable composition comprises about 1 mcg to about 100 mcg of tiotropium bromide.

7. The method of claim 2, wherein the nebulizable composition when administered by the vibrating mesh nebulizer exhibits a delivered dose of about 10% to about 70%.

8. The method of claim 2, wherein the time taken to nebulize the nebulizable composition is about 1 to about 15 minutes.

9. The method of claim 2, wherein the nebulizable composition comprises (a) about 10 to about 80 μg tiotropium bromide, (b) about 18,000 μg of sodium chloride, (c) about 20 μg of disodium edetate, (d) hydrochloric acid, and (e) water, wherein the composition has a pH of about 2.7.

10. The method of claim 2, wherein the nebulizable composition comprises (a) about 10 to about 80 μg tiotropium bromide, (b) about 0.9% w/w sodium chloride, (c) 0.001% w/w disodium edetate, (d) hydrochloric acid, and (e) water, wherein the composition has a pH of about 2.7.

11. The method of claim 2, wherein the nebulizable composition comprises (a) about 10 to about 80 μg tiotropium bromide, (b) about 18,000 μg of sodium chloride, (c) 200 μg of disodium edetate, (d) hydrochloric acid, and (e) water, wherein the composition has a pH of about 2.7.

12. The method of claim 2, wherein the nebulizable composition comprises (a) about 10 to about 80 μg tiotropium bromide, (b) about 0.9% w/w sodium chloride, (c) 0.01% w/w disodium edetate, (d) hydrochloric acid, and (e) water, wherein the composition has a pH of about 2.7.

13. The method of claim 2, wherein the nebulizable composition comprises (a) about 10 to about 80 μg tiotropium bromide, (b) about 18,000 μg of sodium chloride, (c) 400 μg of disodium edetate, (d) hydrochloric acid, and (e) water, wherein the composition has a pH of about 2.7.

14. The method of claim 2, wherein the nebulizable composition comprises (a) about 10 to about 80 μg tiotropium bromide, (b) about 0.9% w/w sodium chloride, (c) 0.02% w/w disodium edetate, (d) hydrochloric acid, and (e) water, wherein the composition has a pH of about 2.7.

15. A method of treating an inflammatory or obstructive airway disease comprising administering via a vibrating mesh nebulizer a nebulizable composition comprising tiotropium bromide to generate an aerosol at a respirable dose delivery rate of about 0.25 μg/min to about 20 μg/min.

16. A method of treating an inflammatory or obstructive airway disease comprising administering via a vibrating mesh nebulizer a nebulizable composition comprising tiotropium bromide to generate an aerosol having one or more of
(i) a geometric standard deviation of emitted droplet size distribution of the nebulizable composition of about 1 to about 3,
(ii) a mass median aerodynamic diameter of droplet size of the nebulizable composition of about 2.5 micron to about 10.5 micron, or
(iii) any combination of any of the foregoing.

17. The method of claim 16, wherein the disease is COPD.

18. The method of claim 16, wherein the aerosol has a droplet size distribution with a D10 of not more than about 5 microns, a D50 of not more than about 10 microns, a D90 of not more than about 20 microns, a Span [(D90−D10)/D50] of not more than about 5, or any combination of any of the foregoing,

19. The method of claim 16, wherein the aerosol has a fine particle dose of not less than 10%.

20. The method of claim 16, wherein the aerosol has a fine particle fraction of about 10% to about 60%.

21. The method of claim 16, wherein the nebulizable composition comprises about 1 mcg to about 100 mcg of tiotropium bromide.

22. The method of claim 16, wherein the nebulizable composition when administered by the vibrating mesh nebulizer exhibits a delivered dose of about 10% to about 70%.

23. The method of claim 16, wherein the time taken to nebulize the nebulizable composition is about 1 to about 15 minutes.

24. The method of claim 16, wherein the nebulizable composition comprises (a) about 10 to about 80 μg tiotropium bromide, (b) about 18,000 μg of sodium chloride, (c) 20 μg of disodium edetate, (d) hydrochloric acid, and (e) water, wherein the composition has a pH of about 2.7.

25. The method of claim 16, wherein the nebulizable composition comprises (a) about 10 to about 80 μg tiotropium bromide, (b) about 0.9% w/w sodium chloride, (c) 0.001% w/w disodium edetate, (d) hydrochloric acid, and (e) water, wherein the composition has a pH of about 2.7.

26. The method of claim 16, wherein the nebulizable composition comprises (a) about 10 to about 80 μg tiotropium bromide, (b) about 18,000 μg of sodium chloride, (c) 200 μg of disodium edetate, (d) hydrochloric acid, and (e) water, wherein the composition has a pH of about 2.7.

27. The method of claim 16, wherein the nebulizable composition comprises (a) about 10 to about 80 μg tiotropium bromide, (b) about 0.9% w/w sodium chloride, (c) 0.01% w/w disodium edetate, (d) hydrochloric acid, and (e) water, wherein the composition has a pH of about 2.7.

28. The method of claim 16, wherein the nebulizable composition comprises (a) about 10 to about 80 μg tiotropium bromide, (b) about 18,000 μg of sodium chloride, (c) 400 μg of disodium edetate, (d) hydrochloric acid, and (e) water, wherein the composition has a pH of about 2.7.

29. The method of claim 16, wherein the nebulizable composition comprises (a) about 10 to about 80 μg tiotropium bromide, (b) about 0.9% w/w sodium chloride, (c) about 0.02% w/w disodium edetate, (d) hydrochloric acid, and (e) water, wherein the composition has a pH of about 2.7.
